(19) United States
(12) Patent Application Publication
(10) Pub. No.: US 2024/0174301 A1
(43) Pub. Date: May 30, 2024

(54) Closure Device and Method for Closing Openings in Carrier Components

(71) Applicant: Illinois Tool Works Inc., Glenview, IL (US)

(72) Inventors: Julia PALOMER, Kaiserslautern (DE); Francisco CRIADO DEL PINO, Cerdanyola del Valles (ES); Marc VISA SCHEIMANN, Calella (ES); Francisco CUERVAS FLORIDO, Rubi (ES); Kevin KIRCHHOFF, Helmstedt (DE)

(21) Appl. No.: 18/522,669

(22) Filed: Nov. 29, 2023

(30) Foreign Application Priority Data
Nov. 30, 2022 (EP) 22210643.7
Nov. 15, 2023 (DE) 10 2023 131 874.8

(51) Int. Cl. B62D 25/24

(52) U.S. Cl. CPC B62D 25/24

(57) ABSTRACT

According to the disclosure, a closure device is provided for closing openings in carrier components, in particular in vehicle bodies of motor vehicles. This comprises a base body, preferably formed of a hard component, for at least partially disposing in an opening of a carrier component having at least one through opening forming a media passage, the base body having a flange section extending parallel to a surface of the carrier component defining the opening; and a sealing element, the sealing element being formed from a material which can be expanded by means of heat, and being arranged on the base body in order, in the expanded state, to completely close off the opening of the carrier component, the closure device having, in particular, means for holding the closure device in the region of the opening of the carrier component.

### RELATED APPLICATIONS

The present application claims the benefit of European Patent Application No. 22210643.7, filed on Nov. 30, 2022, and also German Patent Application No. 10 2023 131 874.8, filed Nov. 15, 2023, the contents of which are hereby incorporated by reference.

### BACKGROUND

In vehicle bodies in particular, a large number of holes and openings are provided to make vehicle cavities, such as the sills, accessible from the outside, in particular to make them accessible for the entry of corrosion protection agents.

Early in the production of a motor vehicle, the body as a whole is usually subjected to phosphating in a phosphating bath. This is followed by washing in a cleaning bath and then usually cataphoretic dip coating. For all these processes, it is necessary for the corresponding liquids to also get into the cavities of a car body. For this purpose, the car body has openings and, in particular, holes that are subsequently to be sealed to prevent dirt and liquids from entering. In addition, vehicle bodies also have holes that are used to manage the production process of an alignment of individual body components until their integration into the overall body.

The known holes or openings are usually closed with rubber-elastic plugs. For this purpose, conventional rubber-elastic plugs have a central cavity in the form of a flat cup-shaped or cup-shaped region which is bounded by a first side wall and a first wall which usually closes off this region on one side.

From U.S. Pat. No. 6,319,436 B1 a plug for closing an opening in a flat surface is known with a plurality of retaining hooks and a circumferential seal, wherein ribs extend from an underside of a transverse surface of the plug, which are formed as guide elements when inserting the plug. The stopper is made of a hard plastic.

A plug for closing holes in flat objects is known from JP 61-59067, said plug being formed of an elastic material and having a first inner circumferential wall which, in one embodiment, has a circumferential step on the outside and a sealing lip cooperating therewith and can accordingly close a hole by these means. In addition, the plug can be corrugated in cross-section with an upper circumferential V-groove in which four ribs are formed.

From US 2015/0135598 A1, a rubber-elastic stopper is known, which consists of a circumferential wall and a top sealing lip formed therefrom, wherein an inner dome-like elevation is arranged on an inner circumferential surface of the wall, the roof of the dome-like elevation being formed with ribs.

EP 2 810 855 A1 discloses a sealing plug for sealing an opening in a component. This has a substantially annular or cylindrical shaft which is interrupted by ribs which are outwardly open and hollow. Furthermore, a sealing lip of conical design is provided on the shaft, at least one flexible folding zone being provided on the sealing lip, which can be reversibly folded in such a way that, in a folded state of the folding zone, the circumference of the sealing lip is reduced in the region of the folding zone and/or of an edge of the sealing lip.

EP 2 781 806 A1 describes a plug for closing openings. This plug comprises a radially circumferential side wall and a sealing surface formed on the side wall, wherein struts can be provided in the region between the circumferential side walls. According to one embodiment, the struts may extend to a circular element concentric with the circumferential side wall, or the struts may end in the region of a central actuating recess formed in an upper wall of the plug.

DE 201 07 612 U1 discloses a closure cap for closing an opening. This closure cap comprises a base body with a circumferential collar which merges into an elastic sealing lip resting against one side of a support. This closure lid may have struts that do not extend from one side of the collar or a radially circumferential sidewall to the other. According to another embodiment, the closure cap may have a grid-like stiffening structure, wherein corresponding interstices of the grid-like stiffening structure are foamed with PVC.

EP 2 873 894 A1 describes a sealing plug for closing an opening. It comprises an annular sealing section and an expanding section which is integral with the sealing section. This sealing plug can also have struts which are bounded approximately in the center of the sealing plug by an element arranged concentrically to the annular sealing section.

DE 10 2009 057750 A1 describes a device for insertion into an opening in a component of an automobile. In this case, a hot-melt adhesive material is applied circumferentially to a flange which, when heated, adheres the flange to a component surface in a sealing manner. The remaining areas of this device are made of a different plastic, whereby this device can be manufactured by means of a 2-component injection molding process.

CN 206 938 651 U discloses a sealing plug manufactured by a 2-component injection molding process, wherein a sealing portion may be formed of a hot-melt material and a base body is formed of a hard component. The sealing body may have a top wall in the connecting portion with the base body, which is formed by thin-walled struts to produce and connect the base body 100 made of the hard component and the sealing body made of the hot-melt material by a 2-component injection molding method.

A sound baffle is disclosed in EP 2 858 883 B1. This comprises: a carrier having a circumferentially extending border substantially surrounding an inner body portion, wherein a plurality of spaced apart members provide void spaces in the inner body portion; and an expandable material containing voids, the expandable material being disposed on the support so that the voids in the expandable material overlie the voids of the support, and in an amount and of such material that it is or they are sufficient for thermal expansion thereto to fill a vehicle cavity and cover the inner body portion and each of the voids, the sound baffle comprising a fastener comprising a clip integrally formed with the carrier, the expandable material extending to a location at least 1 mm from an outer edge of the carrier, the expandable material extending laterally to a portion remote from the outer edge of the carrier.

The object of the present disclosure is to provide a closing device for closing openings in carrier components, in particular in motor vehicles, by means of which an opening in a component can be closed safely and reliably in a simple manner.

A further object of the present disclosure is to provide a closure device for closing openings in carrier components which is of simple construction and provides an alternative to devices known in the prior art.

### SUMMARY

The present disclosure relates generally to a closure device, substantially as illustrated by and described in connection with at least one of the figures, as set forth more completely in the claims. In one example, the present disclosure relates to a closure device and a method for closing openings in carrier components, in particular in motor vehicles.

### BRIEF DESCRIPTION OF THE DRAWINGS

The foregoing and other objects, features, and advantages of the devices, systems, and methods described herein will be apparent from the following description of particular examples thereof, as illustrated in the accompanying figures; where like or similar reference numbers refer to like or similar structures. The figures are not necessarily to scale, emphasis instead being placed upon illustrating the principles of the devices, systems, and methods described herein.

FIG. 1 illustrates a perspective view of a closure device according to the disclosure in accordance with a first embodiment.

FIG. 2 illustrates a side cut perspective view of the closure device entering an opening in a component.

FIG. 3 illustrates a perspective side view of the closure device with expanded sealing element in a final assembly position.

FIG. 4 illustrates a perspective view of the closure device in a pre-assembly position.

FIG. 5 illustrates a perspective partial side view of the closure device in the pre-assembly position.

FIG. 6 illustrates a perspective view of the closure device in a final assembly position.

FIG. 7 illustrates another perspective view of the closure device in a final assembly position.

FIG. 8 illustrates a perspective view of the closure device according to the disclosure according to a second embodiment.

FIG. 9 illustrates another perspective view of the closure device according to the second embodiment.

FIG. 10 illustrates a perspective view of the closure device according to the disclosure according to a third embodiment.

FIG. 11 illustrates a further perspective view of the closure device according to the third embodiment.

### DETAILED DESCRIPTION

References to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from the context. Recitation of ranges of values herein are not intended to be limiting, referring instead individually to any and all values falling within and/or including the range, unless otherwise indicated herein, and each separate value within such a range is incorporated into the specification as if it were individually recited herein. In the following description, it is understood that terms such as “first,” “second,” “top,” “bottom,” “side,” “front,” “back,” and the like are words of convenience and are not to be construed as limiting terms. For example, while in some examples a first side is located adjacent or near a second side, the terms “first side” and “second side” do not imply any specific order in which the sides are ordered.

The terms “about,” “approximately,” “substantially,” or the like, when accompanying a numerical value, are to be construed as indicating a deviation as would be appreciated by one of ordinary skill in the art to operate satisfactorily for an intended purpose. Ranges of values and/or numeric values are provided herein as examples only, and do not constitute a limitation on the scope of the disclosure. The use of any and all examples, or exemplary language (“e.g.,” “such as,” or the like) provided herein, is intended merely to better illuminate the disclosed examples and does not pose a limitation on the scope of the disclosure. The terms “e.g.,” and “for example” set off lists of one or more non-limiting examples, instances, or illustrations. No language in the specification should be construed as indicating any unclaimed element as essential to the practice of the disclosed examples.

The term “and/or” means any one or more of the items in the list joined by “and/or.” As an example, “x and/or y” means any element of the three-element set { (x), (y), (x, y)}. In other words, “x and/or y” means “one or both of x and y”. As another example, “x, y, and/or z” means any element of the seven-element set {(x), (y), (z), (x, y), (x, z), (y, z), (x, y, z)}. In other words, “x, y, and/or z” means “one or more of x, y, and z.”

According to the disclosure, a closure device is provided for closing openings in carrier components, in particular in vehicle bodies of motor vehicles. The closure device comprises a base body, preferably formed of a hard component, for at least partially disposing in an opening of a carrier component having at least one through opening forming a media passage, the base body having a flange section extending parallel to a surface of a component defining an opening; and a sealing element, the sealing element being formed from a material which can be expanded by means of heat, and being arranged on the base body in order, in the expanded state, to completely close off an opening of a carrier component, the closure device having, in particular, means for holding the closure device in the region of an opening of a carrier component.

By means of the sealing device according to the disclosure, an opening of a component can be securely and reliably sealed by expanding the sealing element by means of heat, so that both the media passage and other free spaces between the sealing device and the component are closed.

Via the attachment feature or other means for holding the closure device in the region of an opening of a carrier component, the closure device is arranged or held relative to the carrier component.

In addition, the means for holding the closure device can be used to arrange the closure device in a pre-assembly position on the component. In this way, the closure device can be pre-assembled, for example before a carrier component, for example a vehicle body, is introduced into a cathodic dip bath. After completion of the dip bath, the liquid of the dip bath can run out of the carrier component again through the at least one passage opening and the region between the closure device and the carrier component. During a subsequent heat treatment of a component, the heat-expandable material of the sealing element expands in the furnace such that the opening of a component is sealingly closed.

An EVA can preferably be provided as the sealing element.

The base body may include a tubular section, wherein the flange section is formed on one end of the tubular section, and wherein the tubular section defines at least one central passageway opening of the media passageway.

In the closure device according to the disclosure, it is thus provided that the tubular section is arranged approximately in the region of an opening of a carrier component.

The central passage opening allows a medium to flow through the closure device.

The present disclosure offers the advantage, particularly over simple glued-on closure pads, that they can be mounted in a pre-assembly position prior to cathodic dip-coating and the furnace.

The sealing material is preferably expandable EVA so that it expands and seals the opening.

In addition, the stretchable EVA makes a movement so that the outer surface of the plug's closure device (where it is painted) is nearly flush with a surface of a carrier component.

Several radial, for example two to ten and preferably four, through openings of the media outlet can be formed in the tubular section and/or in the flange section.

Due to a higher number of through openings of the media outlet, it is possible for a higher volume flow to flow through the closure device.

The means for retaining the closure device may be formed as retaining arms formed on the tubular section at an end opposite the flange section, the retaining arms extending radially outward and toward an edge of a carrier component defining an opening.

The retaining arms ensure that the closure device is positioned or also retained at a distance from the opening of a carrier component.

In addition, the locking device can be arranged in a pre-assembly position on a carrier component by means of the retaining arms.

The means for holding the closure device may additionally and/or alternatively be formed as radially outwardly extending latch arms formed radially circumferentially on the tubular section, the latch arms having one or more latch edges formed to hold the closure device in an opening of a carrier component.

The technical effect of the latching arms can be analogous to the technical effect of the holding arms, whereby by means of the latching device the closure device is connected to a carrier component.

By providing several locking edges, on the one hand openings with different diameters can be closed with one and the same closure device and, on the other hand, it is possible to variably define or predefine a pre-assembly position, which can also have an influence on a final assembly position of the closure device.

In the area between the retaining arms and/or the latching arms, free spaces are formed in the pre-assembly position, which are also part of the media passage, so that these also increase the volume flow through the opening.

The expandable material of the sealing element may be disposed on one or more of the following areas of the closure device: an outer jacket wall of the tubular section, and/or an inner jacket wall of the tubular section, and/or a surface of the flange section facing in the direction of the tubular section, and/or one or more surfaces facing in the direction of the flange section of the means for holding the closure device.

According to a method for manufacturing a closure device according to the disclosure, it is provided that the closure device is manufactured by means of a two-component injection molding process.

Furthermore, an assembly is provided according to the disclosure. The assembly comprises a carrier component with an opening, and a closure device described above, wherein the closure device is held in a pre-assembly position by means for holding the closure device spaced from an edge of the carrier component defining the opening so that a media passage is formed between the edge of the carrier component and the closure device, wherein a flange section of the closure device is arranged parallel to a surface of the carrier component, and wherein a sealing element of the closure device made of a heat-expandable material is expanded in a final assembly position such that the opening of the carrier component is completely sealed by the closure device.

The advantages of the assembly according to the disclosure correspond analogously to the advantages described above with reference to the closure device according to the disclosure.

The closure device can have a base body with one or more through openings through which a medium can flow in the pre-assembly position.

In addition, according to the disclosure, a method is provided for sealingly closing an opening of a component with a closure device described above. The method comprises the following steps arranging the closure device in a pre-assembly position in the region of an opening of a carrier component, wherein the closure device is held by means of a means for holding the closure device at a distance from the carrier component such that a medium can flow through in the region between the carrier component and the closure device and/or that a medium can flow through one or more passage openings of a medium passage of the closure device, and preferably arranging a flange section of the closure device spaced apart from and parallel to a surface of a carrier component.

Furthermore, the method may comprise the following steps heating a heat-expandable material of the sealing element of the closure device such that the closure device completely seals an opening of a carrier component in a final assembly position, and preferably arranging the flange section by means of the closure device parallel to a surface of a carrier component, the flange section either directly abutting a surface of a carrier component or being sealingly connected to a surface of the carrier component via the sealing element, preferably slightly spaced apart.

The advantages of the process according to the disclosure correspond analogously to the advantages described above on the basis of the closure device.

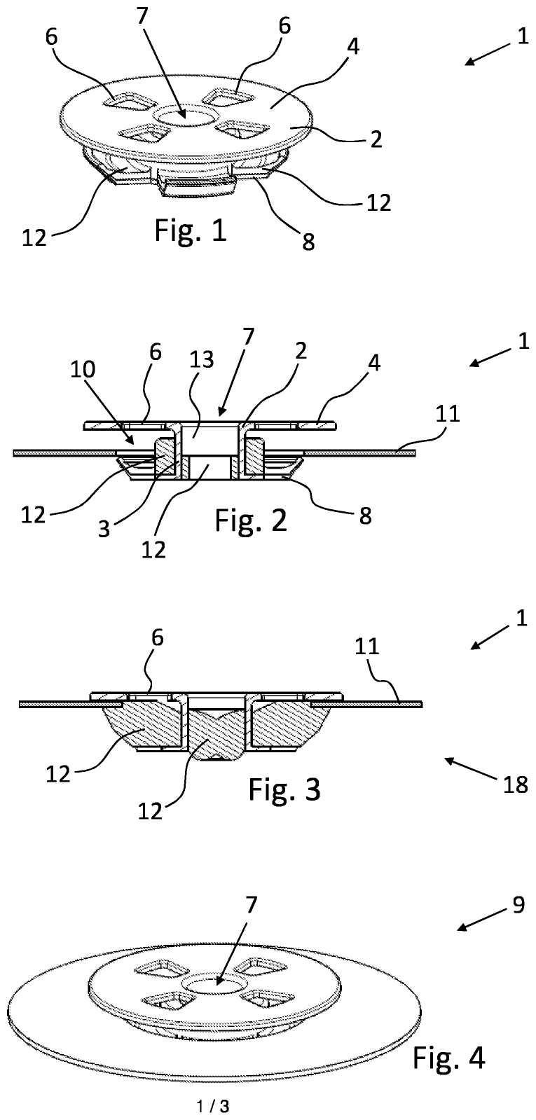

In the following, a closure device 1 according to the disclosure is described in more detail on the basis of a first embodiment (FIGS. 1 to 7). The closure device 1 is designed for closing openings in carrier components, in particular in motor vehicle bodies.

The closure device 1 comprises a base body 2 formed of a hard component.

The base body 2 comprises a tubular section 3. A flange section 4 extending radially outwardly orthogonally to the tubular section 3 is formed on one end of the tubular section 3. Accordingly, the flange section 4 is formed in the shape of a circular ring.

Furthermore, radial passage openings 5 of a media passage 6 are formed radially circumferentially and equally spaced from each other in the flange section.

In addition, the base body 2 delimits a central passage opening 7, which is also part of the media passage 6.

Retaining arms 8 extending radially outward and toward flange section 4 are formed on an end of the tubular section opposite flange section 4.

By means of the retaining arms 8, the locking device can be arranged in a pre-assembly position 9 in an opening 10 of the carrier component 11.

Furthermore, the closure device 1 comprises a sealing element 12. The sealing element 12 is formed of a material that can be expanded by means of heat, such as EVA.

In a non-expanded state, the sealing element 12 is tubularly arranged in the central passage opening 7 or arranged on an inner jacket wall 13 of the tubular section.

Furthermore, the sealing element 12 is arranged on an outer jacket wall 14 of the tubular section 3.

In addition, the sealing element 12 is arranged on surfaces of the retaining arms 8 facing in the direction of the flange section 4.

Accordingly, the closure device 1 according to the first embodiment has two different areas in which the sealing element 12 is applied.

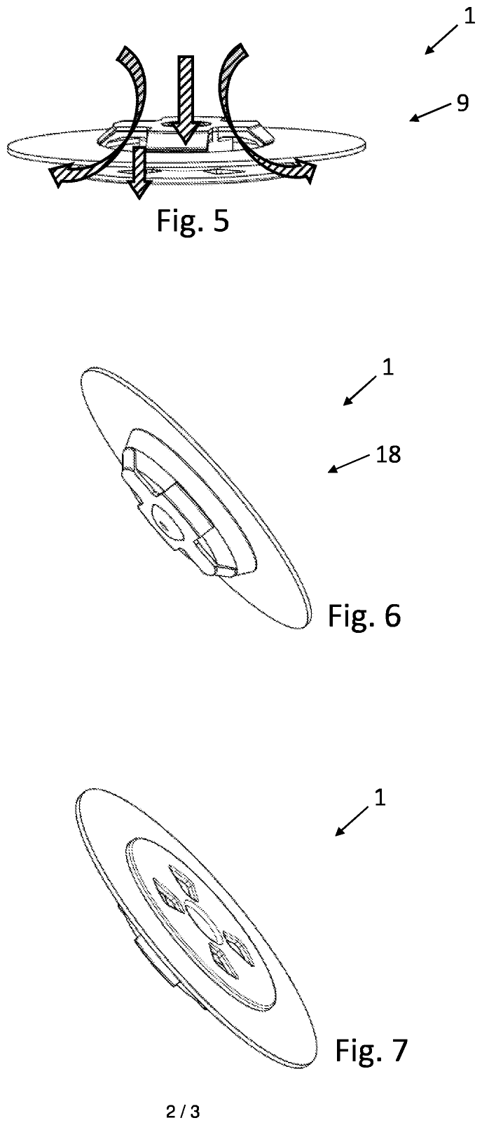

In FIG. 5, the arrows show how the pressure of a liquid causes the closure device to be pressed downwards or towards the carrier component 11 when a vehicle rotates. In this way, the liquid can escape from the component.

FIG. 6 shows how the sealing element made of EVA expands in a final assembly position 18 in the cathodic dip oven and displaces the closure device inward so that an outer surface of the closure device or the flange section is arranged parallel to the surface of the component.

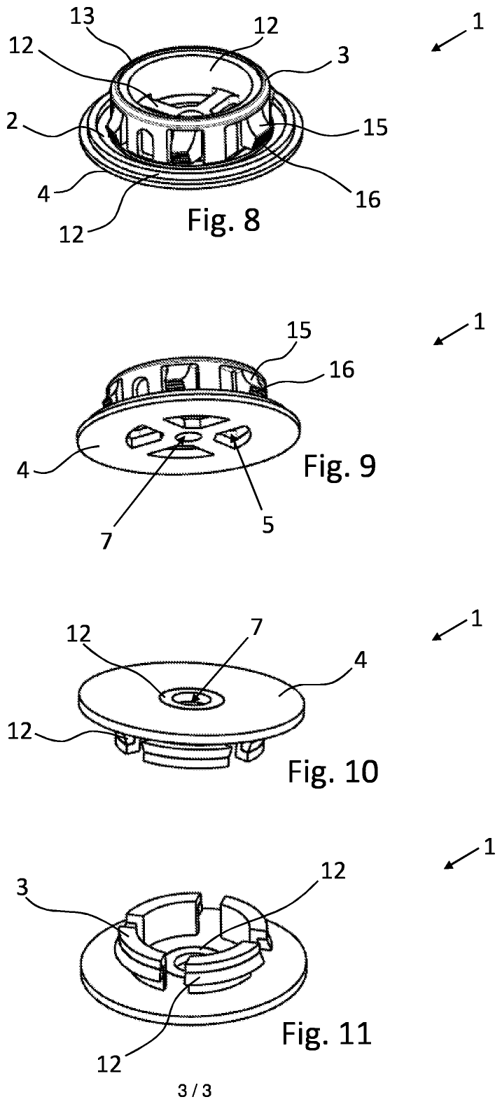

In the following, a closure device 1 according to the disclosure is described in more detail on the basis of a second embodiment example (FIGS. 8 and 9). Unless otherwise described, the closure device 1 according to the disclosure according to the second embodiment example has the same technical features as the closure device 1 according to the first embodiment example. The same technical features are provided with the same reference signs.

According to the second embodiment example, the flange section 4 is disc-shaped or plate-shaped and has, in the region of the tubular section 3, both the central passage opening 7 and four radial passage openings 5 arranged radially circumferentially and equally spaced from one another.

Furthermore, according to the second embodiment example, instead of the retaining arms 8, radially circumferentially arranged latching arms 15 are formed in the jacket wall of the tubular section 3. The latching arms 15 have a plurality of latching edges 16 and extend outward in the radial direction.

The flange section 4 itself of the closure device 1 according to the second embodiment has a smaller surface area than in the first embodiment, with the sealing element 12 being applied to the flange section 4 in the form of a circular ring on the surface facing in the direction of the tubular section 3.

Furthermore, the sealing element 12 is applied to the inner jacket wall 13 of the tubular section 3.

In addition, the sealing element 12 is applied in the areas between the central passage opening 7 and the radial passage openings 5 in the area of the tubular section.

Accordingly, in this embodiment example, the sealing element 12 is also applied to the base body 2 on two different or unconnected areas of the closure device.

In the following, a closure device 1 according to the disclosure is described in more detail on the basis of a third embodiment example (FIGS. 10 and 11). Unless otherwise described, the closure device 1 according to the disclosure according to the third embodiment example has the same technical features as the closure devices 1 according to the first and second embodiment examples. The same technical features are provided with the same reference signs.

According to the third embodiment, the flange section 4 is also disc-shaped or plate-shaped and delimits the central passage opening 7.

The tubular section 3 is formed on the flange section 4, wherein according to this embodiment example the tubular section 3 is segmented in such a way that the jacket wall of the tubular section 3 forms latching arms.

According to this embodiment example, the sealing element 12 is arranged tubularly in the central through opening 7. Furthermore, the sealing element 12 is applied to latching edges 16 of the latching arms 15 of the segmented tubular section 3 facing in the direction of the flange element.

According to all embodiments of the closure device according to the disclosure, it is provided that the hard component or the base body 2 of the closure device 1 is over-molded and manufactured with the sealing element 12 in a 2-component injection molding process.

In addition, an assembly 17 is provided according to the disclosure. The assembly 17 comprises the carrier component 11 with the opening 10 and the closure device 1.

In the pre-assembly position 9, the closure device is held at a distance from an edge of the component defining the opening by means for holding the closure device. The means for holding the closure device can be both the holding arms 8 and the latching arms 15.

Due to the spaced arrangement of the closure device to the component in the pre-assembly position, the media passage 6 is formed between the edge of the component and the closure device.

The flange section 4 of the closure device 1 is arranged parallel to a surface of the carrier component 11.

In addition, the sealing element 12 of the closure device made of the material expandable by means of heat is expanded in a final assembly position 18 in such a way that the opening 10 of the carrier component 11 is completely sealed by means of the closure device. The closure device 1 has the base body 2 in which one or more media passages 6 or through radial and central openings 5, 7 through which a medium can flow in the pre-assembly position 9.

According to a method for manufacturing a closure device according to the disclosure, it is provided that the closure device is manufactured by means of a two-component injection molding process.

Furthermore, an assembly is provided according to the disclosure. The assembly comprises a carrier component with an opening, and a closure device described above, wherein the closure device is held in a pre-assembly position by means for holding the closure device spaced from an edge of the carrier component defining the opening so that a media passage is formed between the edge of the carrier component and the closure device, wherein a flange section of the closure device is arranged parallel to a surface of the carrier component, and wherein a sealing element of the closure device made of a heat-expandable material is expanded in a final assembly position such that the opening of the carrier component is completely sealed by the closure device.

The closure device can have a base body with one or more through openings through which a medium can flow in the pre-assembly position.

In addition, according to the disclosure, a method is provided for sealingly closing an opening of a carrier component with a closure device described above. The method comprises the following steps arranging the closure device in a pre-assembly position in the region of an opening of a carrier component, wherein the closure device is held at a distance from the component by means for holding the closure device in such a way that a medium can flow through in the region between the component and the closure device and/or that a medium can flow through one or more passage openings of a medium passage of the closure device, and preferably arranging a flange section of the closure device spaced apart from and parallel to a surface of a carrier component.

Furthermore, the method comprises the following steps heating a heat-expandable material of the sealing element of the closure device such that the closure device completely seals an opening of a carrier component in a final assembly position, and preferably arranging the flange section by means of the closure device parallel to a surface of a carrier component, wherein the flange section either directly abuts a surface of a carrier component or is sealingly connected to a surface of the carrier component via the sealing element, preferably slightly spaced apart.

The above-cited patents and patent publications are hereby incorporated by reference in their entirety. While the present method and/or system has been described with reference to certain implementations, it will be understood by those skilled in the art that various changes may be made and equivalents may be substituted without departing from the scope of the present method and/or system. In addition, many modifications may be made to adapt a particular situation or material to the teachings of the present disclosure without departing from its scope. For example, block and/or components of disclosed examples may be combined, divided, re-arranged, and/or otherwise modified. Therefore, the present method and/or system are not limited to the particular implementations disclosed. Instead, the present method and/or system will include all implementations falling within the scope of the appended claims, both literally and under the doctrine of equivalents.

### LIST OF REFERENCE SIGNS

1 Closing device
2 Base body
3 Tubular section
4 Flange section
5 Radial through hole
6 Media passage
7 Central passage opening
8 Holding arm
9 Pre-assembly position
10 Opening
11 carrier component
12 Sealing element
13 Inner jacket wall
14 Outer jacket wall
15 Latching arms
16 Latching edge
17 Assembly
18 Final assembly position

What is claimed is:

1. A closure device for closing openings in carrier components in motor vehicles, the closure device comprising:
a base body formed of a hard component, for at least partially disposing in an opening of a carrier component having at least one through opening forming a media passage, the base body having a flange section extending parallel to a surface of a component defining the opening; and
a sealing element, the sealing element being formed of a heat-expandable material and being arranged on the base body to completely close the opening of the carrier component in an expanded state,
wherein the closure device comprises an attachment feature configured to hold the closure device in a region of the opening of the carrier component.

2. The closure device according to claim 1, wherein the base body comprises a tubular section, the flange section being formed on one end of the tubular section, and wherein the tubular section defines at least one central passageway opening of a media passageway.

3. The closure device according to claim 2, wherein a plurality of radial passage openings of a media outlet are formed in the tubular section or in the flange section.

4. The closure device according to claim 2, wherein the attachment feature is formed as holding arms formed on the tubular section at an end opposite the flange section, the holding arms extending radially outwardly and toward an edge of the carrier component defining the opening.

5. The closure device according to claim 2, wherein the attachment feature is designed as radially outwardly extending latching arms which are integrally formed radially circumferentially on the tubular section, the latching arms having one or more latching edges which are designed for holding the closure device in the opening of a component.

6. The closure device according to claim 2, wherein the expandable material of the sealing element is arranged on one or more of the following:
an outer jacket wall of the tubular section;
an inner jacket wall of the tubular section;
a surface of the flange section facing in a direction of the tubular section; or
one or more surfaces facing the flange section of the attachment feature.

7. An assembly comprising,
the carrier component with the opening, and
the closure device according to claim 1,
wherein the closure device is held in a pre-assembly position by the attachment feature spaced from an edge of the carrier component defining the opening so that a media passage is formed between the edge of the carrier component and the closure device,
wherein a flange section of the closure device is arranged parallel to a surface of the carrier component, and
wherein a sealing element of the closure device made of a material expandable via heat is expanded in a final assembly position in such a way that the opening of the carrier component is completely sealed via the closure device.

8. The assembly according to claim 7,
wherein the closure device has a base body with one or more media passages through which a medium can flow in the pre-assembly position.

9. A method of sealingly closing the opening of the carrier component with the closure device according to claim 1, the method comprising:
arranging the closure device in a pre-assembly position in the region of the opening of the carrier component,
wherein the closure device is held by the attachment feature at a distance from the carrier component such that a medium can flow through in the region between the component and the closure device or that a medium can flow through one or more passage openings of the media passage of the closure device; and
arranging a flange section of the closure device spaced apart from and parallel to a surface of a component.

10. The method according to claim 9, wherein the method further comprises:
heating a heat-expandable material of a sealing element of the closure device such that the closure device completely seals the opening of the carrier component in a final assembly position; and
arranging the flange section of the closure device parallel to a surface of the carrier component, the flange section either bearing directly against a surface of the carrier component or being sealingly connected to a surface of the component via the sealing element at a distance therefrom.
